(19) United States
(12) Patent Application Publication
(10) Pub. No.: US 2023/0189901 A1
(43) Pub. Date: Jun. 22, 2023

(54) MASTECTOMY BRA WITH PROSTHESIS

(71) Applicant: Elizabeth D. Liotus, Phoenix, AZ (US)

(72) Inventor: Elizabeth D. Liotus, Huntley, IL (US)

(21) Appl. No.: 18/067,613

(22) Filed: Dec. 16, 2022

Related U.S. Application Data
(60) Provisional application No. 63/290,566, filed on Dec. 16, 2021.

(51) Int. Cl. A41C 3/14; A61F 2/52

(52) U.S. Cl. CPC A41C 3/148; A61F 2/52

(57) ABSTRACT

Embodiments of brassieres with prosthesis for mastectomy patients are described herein. Other embodiments may be described and claimed.

### RELATED APPLICATIONS

### Field

The present disclosure relates generally to a mastectomy bra, and more particularly, a mastectomy bra and prosthesis to be worn after a bilateral or unilateral mastectomy.

### BACKGROUND

Breast cancer affects 1 in 8 women worldwide. For reasons related to a BReast CAncer (BCRA) gene mutation, family history of breast cancer, fear of recurrence, and more, many of these women elect to undergo a mastectomy (one or both breasts removed) without subsequent surgical reconstruction. These women often wear a prosthesis post-procedure, which serves to simulate the natural appearance of healthy breasts through a means that is comfortable and convenient to the wearer. Traditional prostheses include weighted silicon breasts, which can be adhered to the wearer's chest, weighted silicon breasts that are received by a mastectomy bra, or foam inserts that can are received by a mastectomy bra. The traditional prostheses, however, are often unreliable (the adhesive fails), heavy, or difficult to insert. Thus, there is a need in the art for a user friendly mastectomy bra and prosthesis.

### BRIEF DESCRIPTION OF THE DRAWINGS

FIG. 1 shows a front view of a mastectomy brassiere according to a first example.

FIG. 2 shows an exploded view of a front panel according to the first example.

FIG. 3 shows a rear view of the front panel according to the first example.

FIG. 4 shows a rear view of the front panel according to a second example.

FIG. 5 shows a front view the prosthetic breast form according to a first example.

FIG. 6 shows a front view of the prosthetic breast form according to a second example.

FIG. 7A-7B show a front view of the prosthetic breast form according to a first left and right embodiment.

FIGS. 8A-8B show a front view of the prosthetic breast form according to a second left and right embodiment.

FIGS. 9A-9C show a front view of the mastectomy brassiere comprising a lace panel according to alternative embodiments.

FIG. 10 shows a rear view of the front panel according to a first alternative embodiment.

FIG. 11 shows an exploded view of the front panel of FIG. 10.

FIG. 12 shows a rear view of the front panel according to a second alternative embodiment.

FIG. 13 shows an exploded view of the front panel of FIG. 12.

FIG. 14 shows a rear view of the front panel according to a third alternative embodiment.

FIG. 15 shows an exploded view of the front panel of FIG. 14.

FIGS. 16A-16E show a front view of the methodology of putting on the brassiere.

### DEFINITIONS

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The brassiere described herein is intended to be worn by a person wishing to provide an outer appearance of natural breasts. Therefore, the brassiere described herein may not be limited solely to mastectomy recipients. Descriptions herein may include orientation with reference to the human body.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the invention described herein are, for example, capable of operation in other orientations than those illustrated or otherwise described herein.

The terms “upper,” “lower,” “left,” “right,” and the like in the description and in the claims, if any, are used for descriptive purposes in reference to the orientation of the wearer from a standard neutral position, such that “upper” may be proximate the head, “lower” may be proximate the feet, “left” may be proximate the user's general left side and “right” may be proximate the user's general right side.

The term “bilateral” herein is used to describe portion of the invention herein occurring on or affecting both sides of the structure or body.

The term “unilateral” herein is used to describe a portion of the invention herein occurring on or affecting one side of the structure or body.

Various components of the brassiere described herein may be proportionally sized up or down in order to accommodate a wide range of known brassiere measurements. This includes but is not limited to “circumference,” “cup size,” and “band size.”

The invention described herein constitutes a mastectomy bra with easy installation of a foam or compressible prosthesis through an access slot while being worn.

### DESCRIPTION

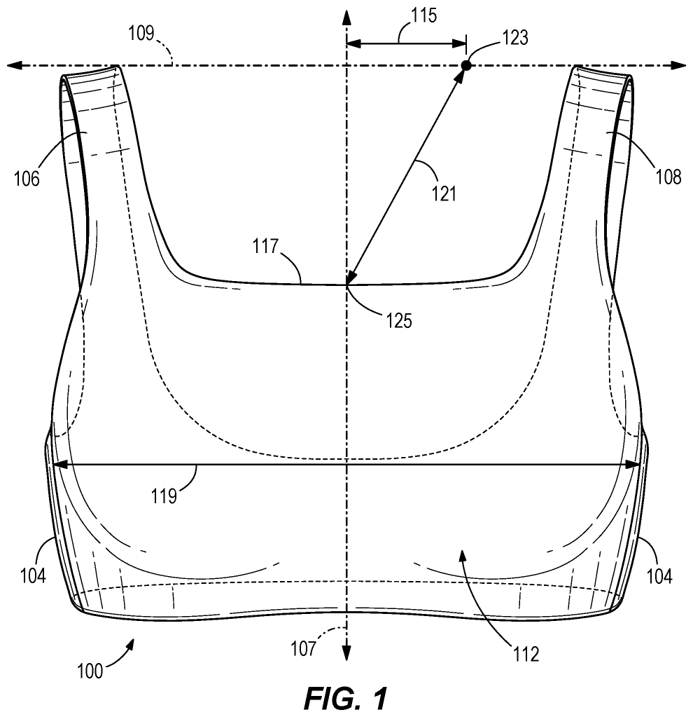

FIGS. 1 & 2 illustrate a first brassiere 100 is shown. The brassiere 100 comprises a front panel 102, a band 104, a first shoulder strap 106, a second shoulder strap 108, and a prosthetic breast form 110. As discussed in more detail below, front panel 102 is configured to be worn over a wearer's chest, spanning armpit to armpit. The band 104 is configured to be joined to the front panel 102 under each armpit and wrap around the wearer's back. The band 104 and front panel 102 cooperate to form an integral brassiere permanently affixed together without a front or back clasp or other bifurcation means. Each of the first and second straps are joined to both the front panel 102 and band 104 and configured to extend over each of the wearer's shoulders. The front panel 102 is further configured to selectively receive and retain the prosthetic breast form 110.

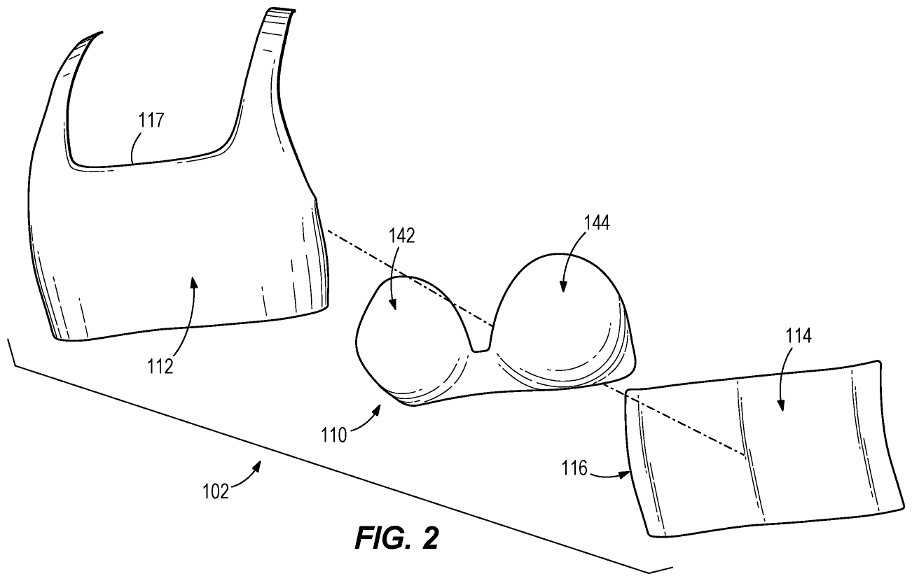

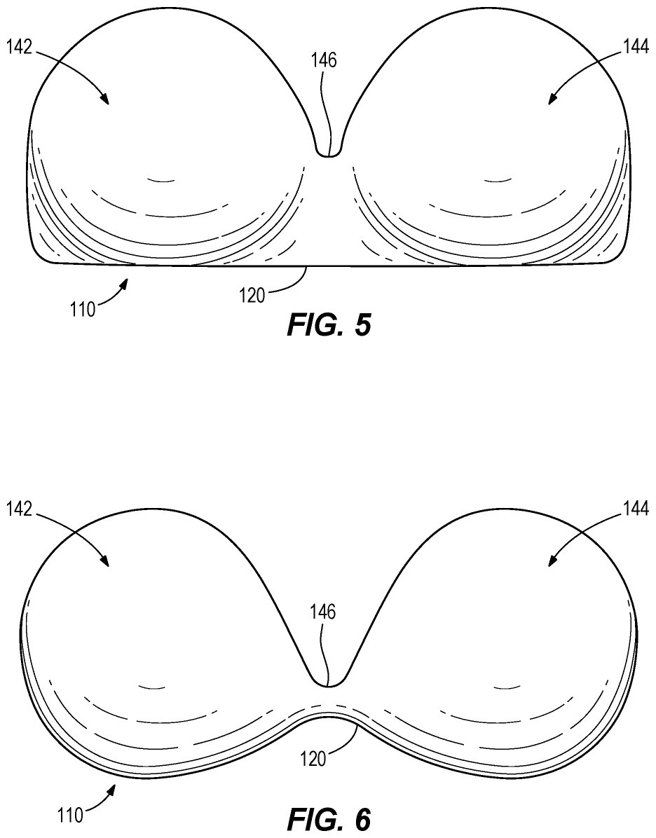

FIG. 2 illustrates an exploded view of brassiere 100. As shown in FIG. 2, front panel 102 further comprises an outer layer 112 and an inner layer 114. The inner layer 114 is configured to be worn adjacent the wearer's chest and the outer layer 112 is configured to face outward adjacent a wearer's shirt. The outer layer 112 and inner layer 114 are overlayed and joined to form a pocket 126 within front panel 102, as shown in FIG. 5. The front panel 102 is formed such that there is no vertical bifurcation or clasping mechanism. The front panel 102 further comprises a perimeter edge 116 along which the outer layer 112 and inner layer 114 are joined. The perimeter edge 116 defines the borders of the pocket 126 which retain the position of the prosthetic breast form 110.

Perimeter edge 116 comprises an upper bust line 118, a lower bustline 120, opposite the upper bust line 118, a first side seam 122, and a second side seam 124, opposite the first side seam. The perimeter edge 116 defines a linear juncture between the outer layer 112 and inner layer 114. Together, the perimeter edge 116, outer layer 112 and inner layer 114 form the borders of the pocket 126 configured to selectively receive and selectively retain the prosthetic breast form 110. The pocket 126 comprises a width that is substantially similar to a width of the prosthetic breast form 110 in order to retain the user's desired position of the prosthetic breast form 110 throughout wear.

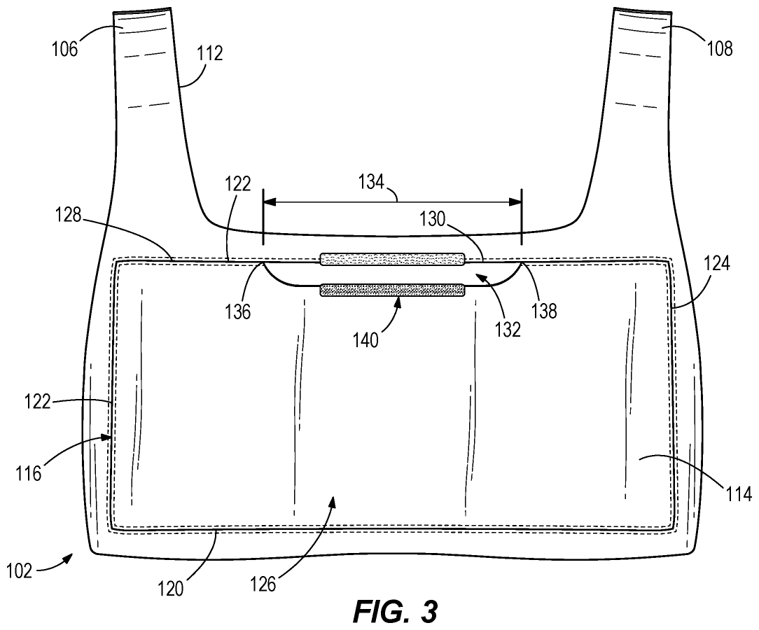

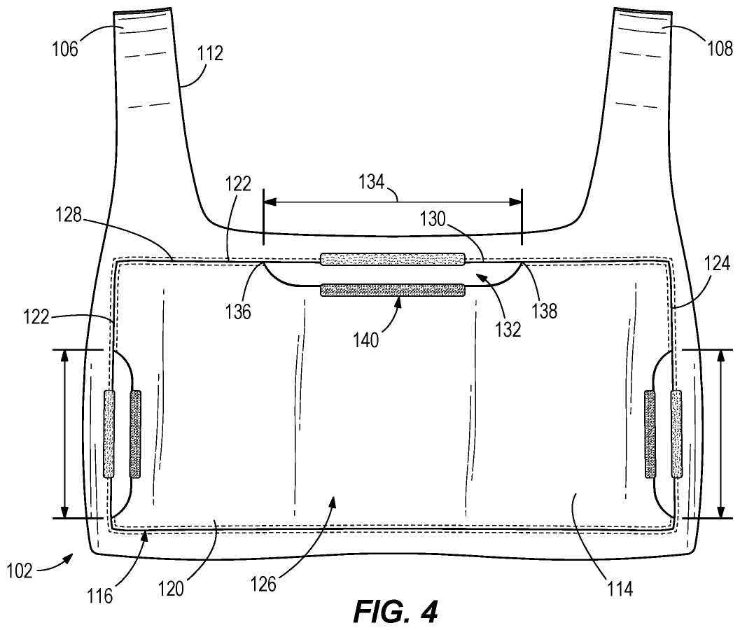

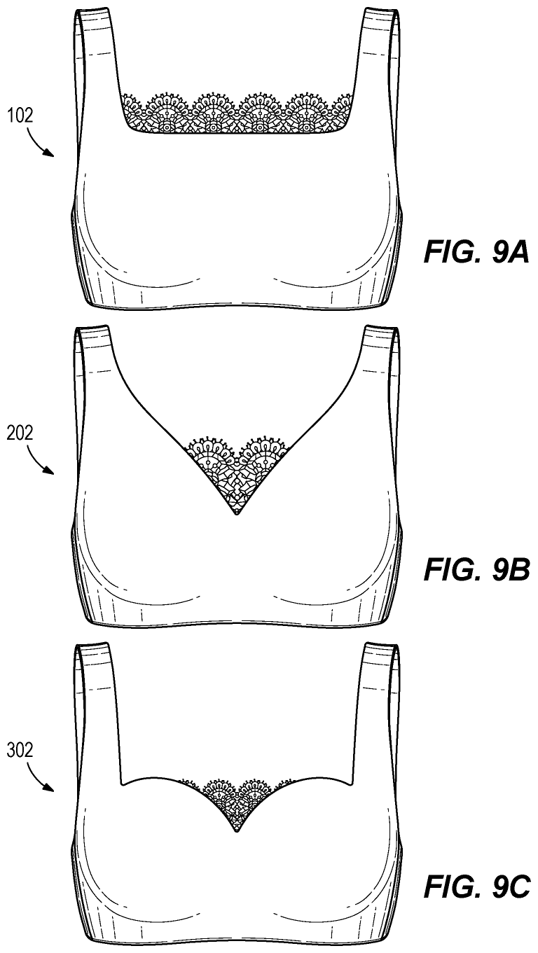

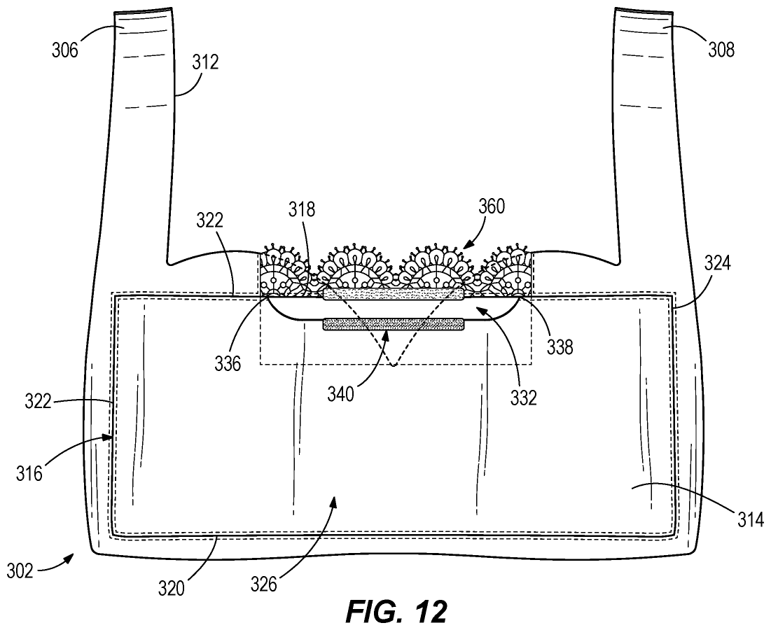

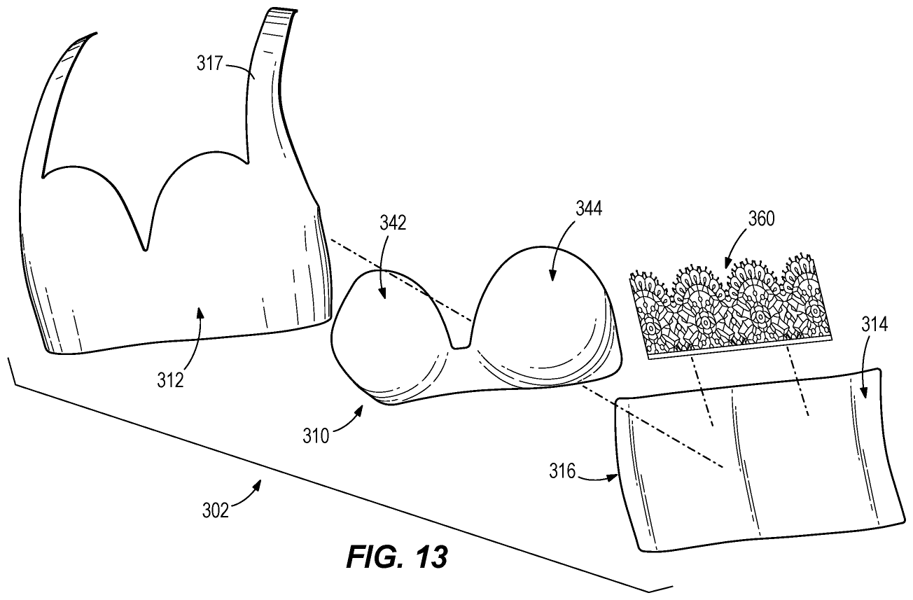

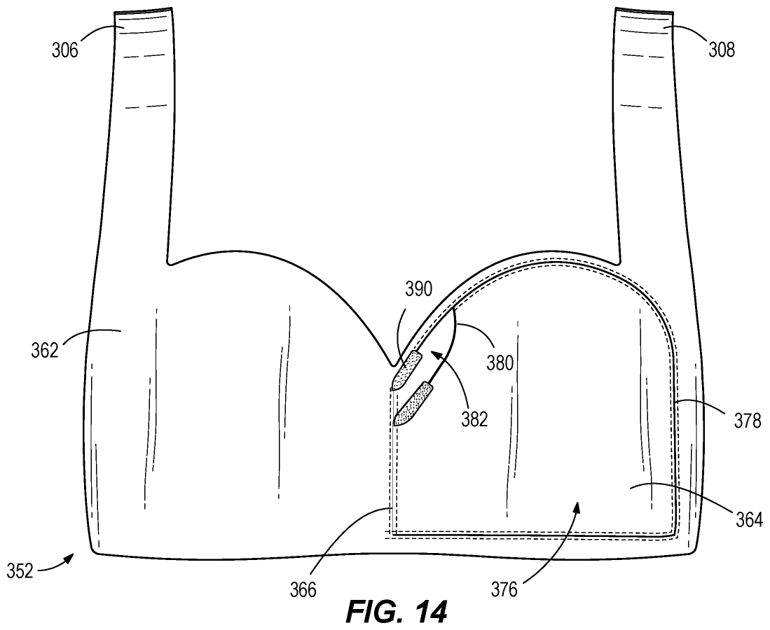

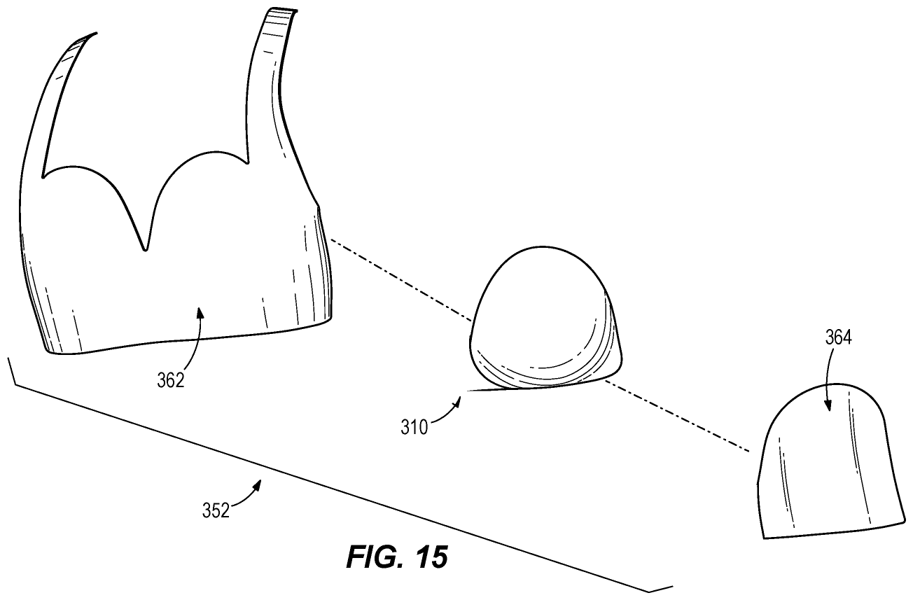

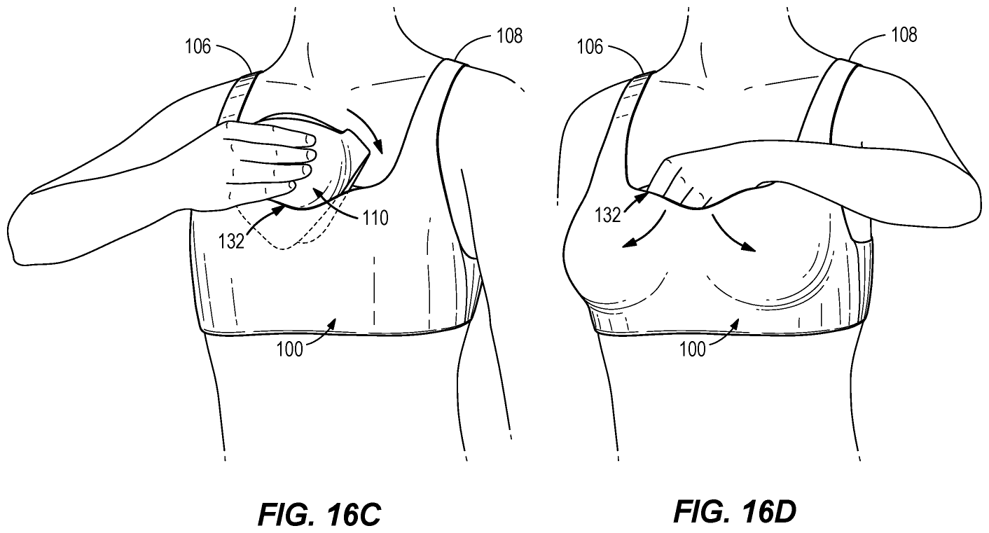

As illustrated in FIGS. 3-4, an upper bustline of the outer layer 112 defines a neckline 117 that describes the shape of the upper bustline 118 and where it cuts across the wearer's body. For example, the upper bustline 118 can comprise a square neckline, scoop neck line, v-neck line, or sweetheart neckline, as illustrated in FIGS. 7A-7B. In the illustrated embodiment, brassiere 100 comprises a square neckline 117. The square neckline 117 is formed by the first shoulder strap 106 and second shoulder strap 108, which meet the upper bustline 118 at a substantially right angle)(˜90°). In other embodiments, the neckline 117 can be a scoop neck. The scoop neck shape can comprise U-shape such that the upper bustline 118 is concave up or curved in an upward direction. In other embodiments, the neckline 117 can be a V-neck having a V-shape. The V-shape is formed when the first shoulder strap 106 and second shoulder strap 108 are angled toward and meet at a center of the bustline 118. Alternatively, the neckline 117 can be a sweetheart neckline, as shown in FIGS. 12-15. The sweetheart neckline comprises bustline 118 that is doubly scalloped. The double scalloped bustline is orientated in a concave down direction, such that the bustline 118 resembles the top half of a heart. In many embodiments, the upper bustline of the perimeter edge 118 is substantially parallel to the neckline 117, as shown in FIGS. 3-4 and 14-15. In alternative embodiments, the upper bustline of the perimeter edge 118 may be straight across rather than comprise a geometry similar to the neckline, as shown in FIGS. 10-13.

The neckline 117 of brassiere 100 comprises a depth 121 to define the position of the upper bustline across the wearer's chest. Depth 121 is defined as the diagonal distance from a shoulder point 123 to an upper bustline center 125. The upper bustline center 125 is defined along a center axis 107. The center axis 107 is defined by half a maximum width 119 of the brassiere and the lower bustline 120, such that center axis 107 passes through a point located at half the maximum width 119 and is perpendicular to the lower bustline 120. As shown in FIG. 1, maximum width 119 is measured parallel to the lower bustline 120 and is measured while the brassiere 100 is laid flat.

The shoulder point 123 represents the joint where the wearer's neck and shoulder meet. The shoulder point 123 is a projected point defined when the brassiere 100 is laid flat and is located along shoulder axis 109. As shown in FIG. 1, shoulder axis 109 is parallel to the lower bustline and passes through a topmost point of the first shoulder strap 106 and second shoulder strap 108. Shoulder point 123 is located at a lateral distance 115 from the center axis 107. The lateral distance 115 of the shoulder point 123 can range from 1.5 inches to 4.00 inches. In many embodiments, the lateral distance 115 of the shoulder point 123 can range inclusively between 1.5 inches and 2.0 inches, 2.0 inches and 2.5 inches, 2.5 inches and 3.0 inches, 3.0 inches and 3.5 inches, or between 3.5 inches and 4.0 inches. For example, the lateral distance 115 of the shoulder point 123 can be 1.5 inches, 1.6 inches, 1.7 inches, 1.8 inches, 1.9 inches, 2.0 inches, 2.1 inches, 2.2 inches, 2.3 inches, 2.4 inches, 2.5 inches, 2.6 inches, 2.7 inches, 2.8 inches, 2.9 inches, 3.0 inches, 3.1 inches, 3.2 inches, 3.3 inches, 3.4 inches, 3.5 inches, 3.6 inches, 3.7 inches, 3.8 inches, 3.9 inches, or 4.0 inches. The shoulder point may alternatively extend beyond the described ranges to accommodate outlier's regarding a person's size and necessary measurements. That is to say the shoulder point 123 may be greater than 4.0 inches or less than 1.5 inches if need be in order to accommodate a person's size.

In an alternative embodiment, the shoulder point can define an angle 150 between the depth 121 of the neckline 117 and the center axis 107 at the upper bustline center 125. The value of the angle 150 can determine the lateral distance 115 of the shoulder point 123. The angle can range between 15 and 45 degrees. In many embodiments, the angle 150 can range inclusively between 15 and 20 degrees, 20 and 25 degrees, 25 and 30 degrees, 30 and 35 degrees, 35 and 40 degrees, or between 40 and 45 degrees. For example, the angle 150 can be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 degrees.

The depth 121 of the neckline 117 is configured to accommodate a variety of styles of clothing, such that the brassiere 100 can be discretely worn under clothing. The neckline depth 121 is measured between shoulder point 123 and the upper bustline center 125 when the brassiere 100 is laid flat. In some embodiments, the depth 121 ranges from 6.00 inches to 12.00 inches. For example, the depth 121 can be 6.00 inches, 6.50 inches, 7.00 inches, 7.50 inches, 8.00 inches, 8.50 inches, 9.00 inches, 9.50 inches, 10.00 inches, 10.50 inches, 11.00 inches, 11.50 inches, or 12.00 inches. The value of depth 121 can be associated with the size of the brassiere 100, such that depth 121 is proportional to the size. That is to say the larger the brassiere 100, the greater the value of the depth 121.

In some embodiments, selective retention of the prosthetic breast form 110 is achieved by the perimeter edge 116. In these embodiments, perimeter edge 116 further comprises a first portion 128 and a second portion 130. As shown in FIG. 3, the first portion 128 of the perimeter edge extends continuously along the lower bust line 120 and first and second side seams 122, 124 and discontinuously across the upper bust line 118. The second portion 138 of the perimeter edge creates the discontinuity of the first portion 128 along the upper bust line 118. Together, the first portion 128 and second portion 130 define the complete perimeter edge 116 of the front panel 102.

As mentioned above, the outer layer 112 and inner layer 114 are joined via a linear juncture along the perimeter edge 116 to form the pocket 126. In some embodiments, selective retention of the prosthetic breast form 110 is further achieved by the linear juncture along the first portion 128 and second portion 130 of the perimeter edge 116. In these embodiments, the linear juncture along the first portion 128 can be permanent and the linear junction along the second portion 130 can be temporary, or adjustable between an open and closed configuration. In other words, the second portion 130 defines an access slot 132 through which the prosthetic breast from can be inserted into the pocket 126. The first portion 128 and second portion 130 follow the geometry or configuration of the outer layer 112 and inner layer 114 perimeter, or the neckline, to define the access slot. The linear junction of the second portion 130 can comprise an open configuration for receiving and removing the prosthetic breast form 110 and a closed configuration for retaining the prosthetic breast form 110 within the pocket 126.

The second portion 130 of the perimeter edge can comprise a fastening mechanism 140 between the outer layer 112 and inner layer 114. The fastening mechanism 140 defines the open and closed configurations of the access slot 132. In some embodiments, the fastening mechanism 140 comprises a male portion, permanently secured to one of the outer layer 112 and the inner layer 114, and a female portion permanently secured to the other of the outer layer 112 and inner layer 114. The male portion and female portion of the fastening mechanism 140 can be selectively engaged and disengaged to allow for insertion and removal of the prosthetic breast form 110. In some embodiments, the fastening mechanism is a hook and loop fastener (e.g., VELCRO®). In other embodiments, the fastening mechanism is buttons, snaps, magnets, eye-hooks, zippers, ties, or any other suitable means for selectively securing the outer and inner layers of the front panel.

The outer layer 112 and inner layer 114, as mentioned above, can be permanently joined along the first portion 128 of the perimeter edge. In some embodiments, the outer and inner layers can be joined via stitching, adhesives, fabric fusion techniques, or any other suitable method for permanently joining textiles. Permanent joining of the outer layer 112 and inner layer 114 along the first portion 128 can ensure the structural integrity of the pocket 126 over repeated washing and user wear. In some embodiments, the second portion 130 of the perimeter edge lacks a fastening mechanism and the prosthetic breast form 110 is secured within the pocket by the surrounding connection of the outer layer 112 and inner layer 114.

In some embodiments, the second portion 130 of the perimeter edge 116 comprises a length 134 of access slot 132. The access slot length 134 can be measured as the horizontal distance between a first point of discontinuity 136 and a second point of discontinuity 138 of the first portion 128 of the perimeter edge. The access slot length 134 is further measured between the first point and second point of discontinuity 136, 138 in an unstretched, or unworn configuration, such that the slot 132 is not distorted. The access slot length 134 can range between 4.00 inches and 7.00 inches. For example, the access slot length 134 can be between 4.00 inches and 4.25 inches, 4.25 inches and 4.50 inches, 4.50 inches and 4.75 inches, 4.75 inches to 5.00 inches, 5.00 inches to 5.25 inches, 5.25 inches to 5.50 inches, 5.50 inches to 5.75 inches, 5.75 inches to 6.00 inches, 6.00 inches to 6.25 inches, 6.25 inches to 6.50 inches, 6.50 inches to 6.75 inches, and 6.75 inches to 7.00 inches. The access slot length 134 can be 4.00 inches, 4.10 inches, 4.20 inches, 4.30 inches, 4.40 inches, 4.50 inches, 4.60 inches, 4.70 inches, 4.80 inches, 4.90 inches, 5.00 inches, 5.10 inches, 5.20 inches, 5.30 inches, 5.40 inches, 5.50 inches, 5.60 inches, 5.70 inches, 5.80 inches, 5.90 inches, 6.00 inches, 6.10 inches, 6.20 inches, 6.30 inches, 6.40 inches, 6.50 inches, 6.60 inches, 6.70 inches, 6.80 inches, 6.90 inches, or 7.00 inches. Material properties of the front panel 102 can allow the access slot 134 to stretch to larger dimensions in order to accommodate insertion of the prosthetic breast form 110.

In some embodiments, the perimeter edge can comprise more than one access slot, as shown in FIG. 4. For example, the perimeter edge can further comprise a second access slot 152 at the perimeter edge first side seam 122 and a third access 162 slot at the perimeter edge second side seam 124 to define one or more side access slots. The side access slots can allow for further adjustment of the brassiere once it is placed within the pocket 126 via the second portion 130 of the perimeter edge. The side access slots can further comprise fastening mechanisms between the outer layer 112 and inner layer 114 similar to fastening mechanism 140. The fastening mechanisms define the open and closed configurations of the side access slots. The fastening mechanisms can be selectively engaged and disengaged to allow for adjustment of the prosthetic breast form 110. In some embodiments, the fastening mechanism is a hook and loop fastener (e.g., VELCRO®). In other embodiments, the fastening mechanism(s) is buttons, snaps, magnets, eye-hooks, zippers, ties, or any other suitable means for selectively securing the outer and inner layers 112, 114 of the front panel 102.

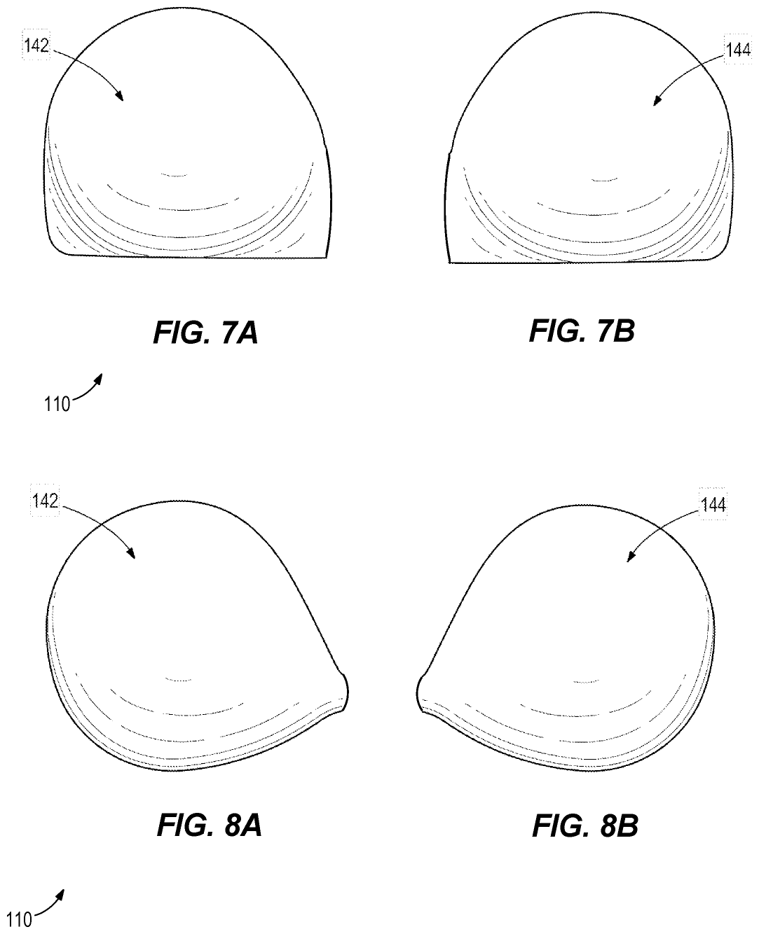

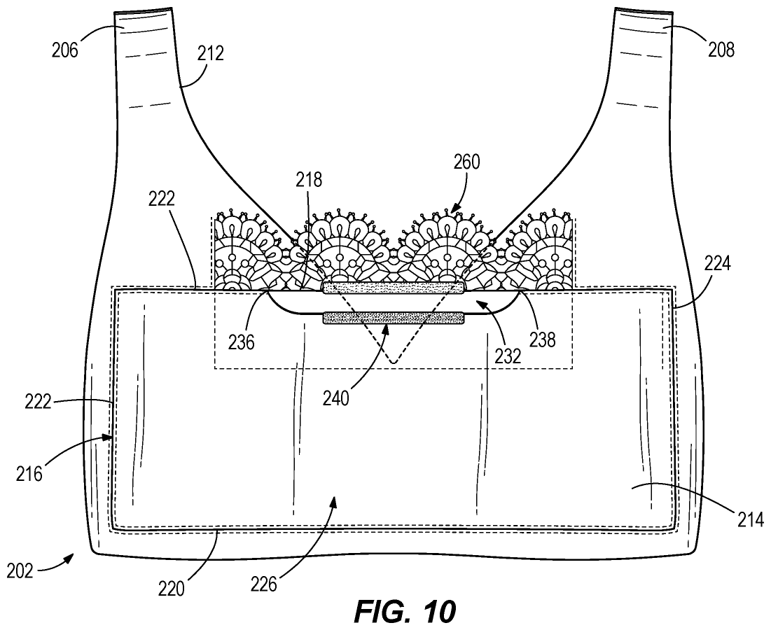

FIGS. 5-6, 7A-7B, and 8A-8B show prosthetic breast form 110 removed from pocket 126. Prosthetic breast form 110 can be a single component comprising a first cup 142 and a second cup 144 connected by a gapping portion 146. The first cup 142 and second cup 144 are configured to shape front panel 102 with the contours of two natural breasts with a single prosthesis. The prosthetic breast form 110 can comprise a shape that renders a visually similar appearance to natural breast(s) once in the brassiere, but able to be manipulated or folded easily for the wearer to readily insert the prosthetic breast form 110 into the pocket 126. For example, as shown in FIG. 5, the prosthetic breast form 110 can comprise a first cup 142 and second cup 144 that are substantially round with a gapping portion 146 that also varies in size according to the size of the first and second cup 142, 144. In an alternative embodiment, the prosthetic breast form 110 can comprise a first cup 142 and a second cup 144 that each form a tear-drop shape, such that the tapered portions of each cup meet at the gapping portion 146, as shown in FIG. 6. It is advantageous for the gapping portion to comprise a lesser volume and length than the cups in order to promote flexure at this location and further provide ease of insertion into the pocket 126.

In alternative embodiments, the prosthetic breast form 310 can comprise a single cup configured to shape front panel 302 with the contour of a single natural breast. Similar to prosthetic breast form 110, the prosthetic breast form 310 can comprise a shape that renders a visually similar appearance to a natural breast once in the brassiere, but able to be folded or manipulated easily for the wearer to readily insert the prosthetic breast form 310 into the pocket 376. For example, as shown in FIGS. 7A-B and 8A-B, the prosthetic breast form 310 can comprise a single cup that is substantially round or tear-drop shaped. The prosthetic breast form 310 can comprise an asymmetrical orientation such that it imitates the appearance of a natural left breast or a right breast. In other embodiments, the prosthetic breast form 310 is symmetrical and able to be used interchangeably as a form for either a left or right breast.

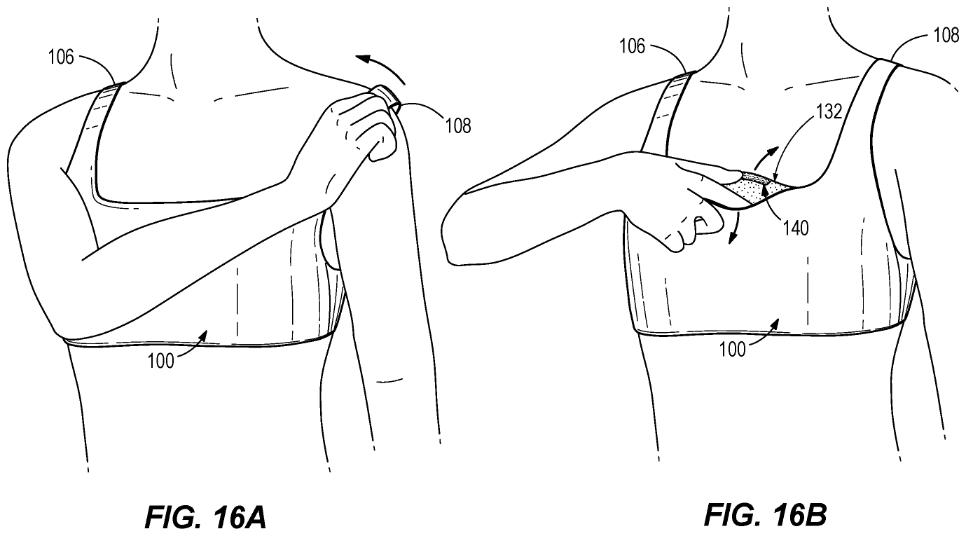

In an alternative embodiment, and similar to the front panel 102, the front panel 352 can comprise a first shoulder strap 306, a second shoulder strap 308, an outer layer 362, and an inner layer 364. The outer layer 362 and inner layer 364 are joined via a linear juncture along a perimeter edge 366 to form the pocket 376, as illustrated in FIG. 14. The inner layer 364 can comprise a length that is approximately half the length of the outer layer 362 to form a pocket 376 that accommodates the size of a prosthetic breast form 310 comprising a single cup. In some embodiments, selective retention of the prosthetic breast form 310 is further achieved by the linear juncture along the first portion 378 and second portion 380 of the perimeter edge 366. In these embodiments, the linear juncture along the first portion 378 can be permanent and the linear junction along the second portion 380 can be temporary, or adjustable between an open and closed configuration. The inner layer 364 can be permanently joined along the first portion 378 on either the left or right side of the outer layer 362. Similar to pocket 126, the second portion 380 defines an access slot 382 through which the prosthetic breast from 310 can be inserted into the pocket 356. The first portion 378 and second portion 380 follow the geometry or configuration of the outer layer 362 and inner layer 364 perimeter, or the neckline, to define the access slot 382. The linear junction of the second portion 380 can comprise an open configuration for receiving and removing the prosthetic breast form 310 and a closed configuration for retaining the prosthetic breast form 310 within the pocket 376. In some embodiments, the second portion 380 of the perimeter edge can further comprise a fastening mechanism 390 similar to fastening mechanism 140.

In many embodiments, the cups 142, 144, 344 described herein can comprise a volume relative to a cup and band size known in the industry.

In some embodiments, prosthetic breast form 110, 310 is constructed from a lightweight polyester foam material. The prosthetic breast form can comprise a front side facing outward from the user's body and a rear side facing inward toward a user's body. The rear side of the prosthetic insert 110, 310 can comprise a substantially flat or slightly concave surface to rest flat against the user's chest. The foam material of the prosthetic breast form can extend from the rear side to the front side to form the appearance of natural breasts. The polyester foam breast form 110, 310 can have a mass ranging between 40 g and 80 g. For example, the prosthetic breast form 110, 310 can have a mass of 40 g, 42 g, 44 g, 46 g, 48 g, 50 g, 52 g, 54 g, 56 g, 58 g, 60 g, 62 gg, 64 g, 68 g, 70 g, 72 g, 74 g, 76 g, 78 g, or 80 g. The mass of prosthetic breast form 110, 310 can define a percentage of a total mass of brassiere 100, 310. The percent mass of the prosthetic breast form 110 can range from 50% to 70% the total mass of the brassiere. For example, the percent mass of the prosthetic breast form 110 can be 50%, 51%, 52%, 53%, 54%, 55%, 56%, 57%, 58%, 59%, 60%, 61%, 62%, 63%, 64%, 65%, 66% 67%, 68%, 69%, or 70%. The percent mass of the prosthetic breast form 310 can range from 25% to 40% the total mass of the brassiere. For example, the percent mass of the prosthetic breast form 310 can be 25%, 26%, 27%, 28%, 29%, 30%, 31%, 32%, 33%, 34%, 35%, 36%, 37%, 38%, 39%, or 40%. In most embodiments, the prosthetic breast form 110 will not be constructed from an incompressible material such as silicon, plastic, or water-filled polymers and the like.

In some embodiments, the front panel 102, band 104, first shoulder strap 106, and second shoulder strap 108 are formed from the same textile material and configured to stretch to fit the wearer's body. The textile material forming front panel 102, band 104, and first and second shoulder straps 106, 108 are formed from a spandex fiber blend. The spandex fiber blend is a lightweight material and comprises a fabric, or textile weight measured in grams per meter (GSM). In some embodiments, the spandex fiber blend is less than or equal to 250 GSM. For example, the spandex fiber blend can have a weight ranging from 150 GSM to 250 GSM. The weight of the spandex fiber blend can be 150 GSM, 155 GSM, 160 GSM, 165 GSM, 170 GSM, 175 GSM, 180 GSM, 185 GSM, 190 GSM, 195 GSM, 200 GSM, 225 GSM, or 230 GSM, 235 GSM, 240 GSM, 245 GSM, or 250 GSM.

The spandex fiber blend can comprise synthetic fibers such as spandex, nylon, polyester, or any other suitable textile fiber. In some embodiments, the spandex fiber blend comprises spandex and nylon. For example, the material can have a 65-95% nylon percentage and a 5-35% spandex percentage. The material can comprise 65% nylon and 35% spandex, 70% nylon and 30% spandex, 75% nylon and 25% spandex, 80% nylon and 20% spandex, 85% nylon and 15% spandex, 90% nylon and 10% spandex, or 95% nylon and 5% spandex. In other embodiments, the nylon composition can have a range between 90% and 95% and spandex composition can have a range between 5% and 10%.

The spandex fiber blend further defines a stretch percentage calculated as the percent change in the horizontal and vertical dimensions of a material, when stretched to a maximum. The horizontal stretch percentage of the spandex fiber blend can be at least 50% and the vertical stretch percentage of the spandex fiber blend can be at least 50%. For example, the spandex fiber blend can have a 50% horizontal stretch and 50% vertical stretch, 65% horizontal stretch and 65% vertical stretch, 70% horizontal stretch and 70% vertical stretch, 75% horizontal stretch and 75% vertical stretch, 80% horizontal stretch and 80% vertical stretch, 85% horizontal stretch and 85% vertical stretch, 90% horizontal stretch and 90% vertical stretch, 95% horizontal stretch and 95% vertical stretch, or 100% horizontal stretch and 100% vertical stretch. In some embodiments, the horizontal stretch and vertical stretch percentages of the spandex fiber blend are equal. In other embodiments, the spandex fiber blend has a greater horizontal stretch percentage than vertical stretch percentage. For example, the spandex fiber blend can have a 100% horizontal stretch percentage and a 50% vertical stretch percentage. The spandex fiber blend has sufficient stretch as to securely retain the position of the prosthetic breast form 110 without crushing or compressing the foam material.

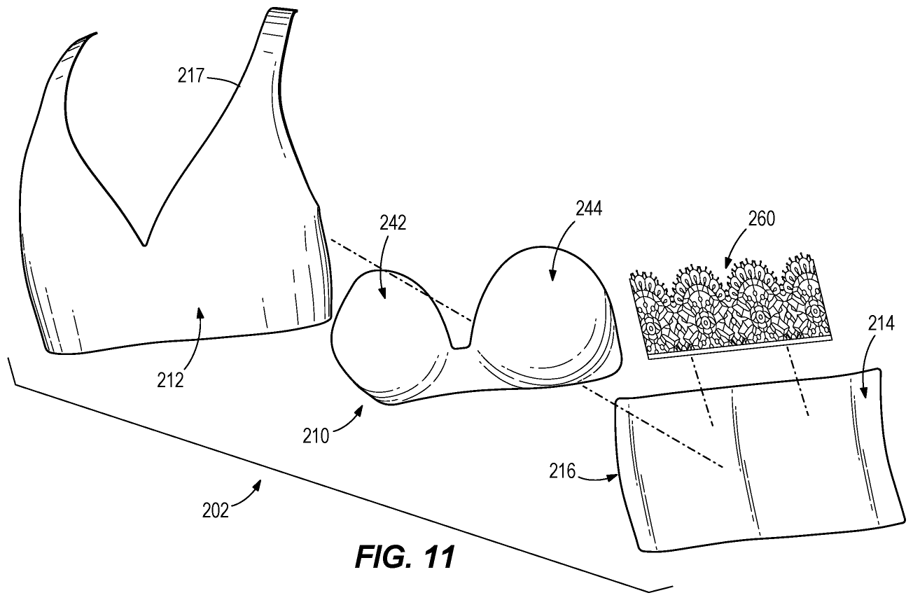

In some embodiments, such as those in FIGS. 9A-9C, the brassiere 100 can further comprise a lace panel 160. The front panel 102, 202, 302 can comprise similar features to front panel 102, further comprising a lace panel, as shown in FIGS. 9A-13. The lace panel 160, 260, 360 can comprise a length that is equal to or less than the length of the front panel 102, 202, 302. The lace panel 160, 260, 360 can be sewn to the front panel 102, 202, 302 in such a way that a top portion of the lace panel 160, 260, 360 is visible above the neckline 117, 217, 317 or defines at least a portion of the neckline 117, 217, 317. In some embodiments, the lace panel 160, 260, 360 is sewn or attached via hook and loop fastener (e.g., VELCRO®) to the front panel 102, 202, 302 on a side that is not in contact with the wearer's skin. In other embodiments any suitable self-fastener or removable fastener may be used, including a button and button hole, a hook-and-eye closure, or a snap fastener. In alternative embodiments, the lace panel 160, 260, 360 is sewn or attached to the front panel 102, 202, 302 on an inner side, in such a way that the lace attachment does not create a visible lump or line under the wearer's clothes. In either embodiment, the lace panel 160, 260, 360 is securely attached to the brassiere 100 without being in direct contact with the wearer's skin or creating visible lumps under user clothing. The lace panel 160, 260, 360 of any of the above embodiments may also be placed or connected to the front panel 102, 202, 302 in such a way that it is completely hidden from exterior view.

### Methodology

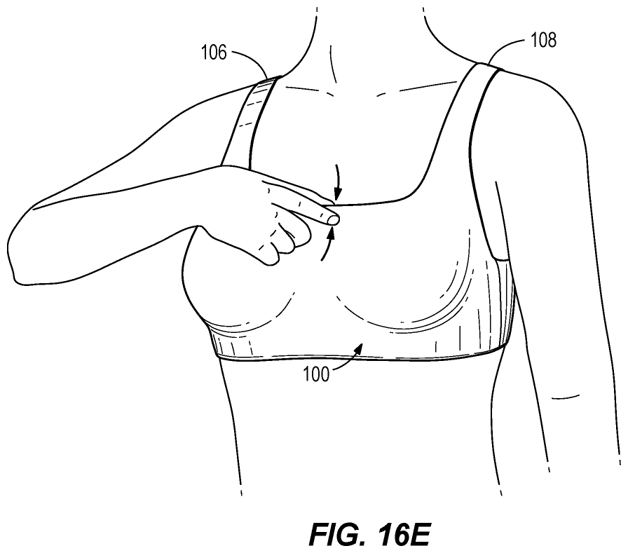

The user can pull the brassiere 100 over their head and place one arm through the first shoulder strap 106 and place a second arm through the second shoulder strap 108 such that the front panel 102 is adjacent the user's chest and the band 104 lies under each armpit and wraps around the user's torso, as shown in FIG. 16A. While wearing the brassiere 100, the user can open the fastening mechanism 140 to place the access slot 132 in an open configuration, if applicable, as illustrated in FIG. 16B. The user can then take the prosthetic breast form 110 and compress or fold it, as shown in FIG. 16C, then slide the prosthetic breast form 110 through the access slot 132 into the pocket 126. As further illustrated in FIG. 16D, the user can then adjust the prosthetic breast form 110 to the desired position through the access slot 132. The user can then close the fastening mechanism 140 of the access slot 132 to the closed configuration, if applicable, as shown in FIG. 16E.

### Advantages

The lightweight prosthetic breast form 110, comprising 50-70% of the total mass, permits greater stretch percentages from the spandex fiber blend than fabrics used to construct brassieres configured to support natural breasts or heavier prosthetics. The horizontal and vertical stretch percentages of brassieres configured to support natural breasts and heavier prosthetics are lower and confined to a range between 30% and 50%.

Further, the larger stretch percentages allow the wearer to put on the brassiere overhead and prior to insertion of the breast form. Thus, brassiere 100 eliminates the need to bifurcate and include a clasping mechanism along the wearer's back. Elimination of the clasping mechanism and the overall greater stretch result in a brassiere that is less tight and less restrictive, such that the brassiere can be worn without irritation to scar tissue or irritation to sensitive skin.

As mentioned above, the prosthetic breast form 110 can be inserted into the pocket 126 while being worn. Together, the depth 121 of the neckline, the stretch properties of the spandex fiber blend, and the larger access slot 132 located along the upper bustline 118 allow the wearer to insert the breast form 110 into the pocket while wearing the brassiere 100. The brassiere 100 prevents the form 110 from twisting or becoming misshaped while pulling the brassiere overhead. Brassiere 100, therefore, significantly improves wearer experience by preventing the frustration of folded or twisted forms, difficult to reach access slots, or access slots that are stretched out due to forcing a folded insert into them.

### Clauses

Clause 1: A brassiere comprising: a front panel; a band; a first shoulder strap; a second shoulder strap; and a prosthetic breast form; wherein: the front panel further comprises an outer layer and an inner layer joined along a first portion of a perimeter edge; the outer layer and inner layer are permanently joined along the first portion of the perimeter edge and define a pocket configured to receive the prosthetic breast form; the outer layer and inner layer are selectively fastenable along a second portion of the perimeter edge to define an access slot to the pocket; wherein: the band is configured to be non-removably joined to the front panel under each of a user's armpits and wrap around the user's back; and each of the first and second shoulder straps are joined to both the front panel and band and configured to extend over each of the user's shoulders.

Clause 2: The brassiere of clause 1, wherein: the prosthetic breast form comprises a first cup, a second cup, and a gapping portion.

Clause 3: The brassiere of clause 1, wherein: the prosthetic breast form comprises a percent mass of the total prosthetic brassier and said percent mass is 50%-70%.

Clause 4: The brassiere of clause 1, wherein: the band lacks a fastening mechanism.

Clause 5: The brassiere of clause 1, wherein: a length of the access slot is between 4.0 inches and 7.0 inches.

Clause 6: The brassiere of clause 1, wherein: the front panel lacks a vertical bifurcating mechanism.

Clause 7: The brassiere of clause 1, wherein: the front panel, the first shoulder strap, the second shoulder strap, and the band comprise a textile material; and the textile material is the same for each the front panel, the first shoulder strap, the second shoulder strap, and the band.

Clause 8: The brassiere of clause 1, wherein: the prosthetic breast form can be inserted into the pocket while the brassiere is being worn by the user.

Clause 9: The brassiere of clause 1, wherein: the prosthetic breast form comprises a compressible material such that a volume of the prosthetic breast form can be reduced while being inserted into the pocket.

Clause 10: The brassiere of clause 1, wherein: the front panel further comprises a lace panel.

Clause 11: The brassiere of clause 9, wherein: the front panel is coupled to the inner layer on a side distal from the user's body.

Clause 12: The brassiere of clause 1, wherein: the front panel further comprises a neckline with a shape chosen from a group consisting of square, V-neck, scoop, or sweetheart.

Clause 13: The brassiere of clause 1, wherein: the prosthetic breast form comprises a flat bottom portion.

Clause 14: A brassiere comprising: a front panel; a band; a first shoulder strap; a second shoulder strap; and a prosthetic breast form; wherein: the band is non-removably joined to the front panel under each of a user's armpits and wraps around the users body such that the front panel is adjacent to the user's chest; the front panel further comprises an outer layer and an inner layer joined along a first portion of a perimeter edge; the outer layer and inner layer are permanently joined along the first portion of the perimeter edge and define a pocket configured to receive the prosthetic breast form; the outer layer and inner layer are not joined along a second portion of the perimeter edge to define an access slot to the pocket; and wherein the prosthetic breast form can be inserted into the pocket through the access slot while the user is wearing the brassiere.

Clause 15: The brassiere of clause 14, wherein: the prosthetic breast form comprises a single cup.

Clause 16: The brassiere of clause 14, wherein: the prosthetic breast form comprises a first cup, a second cup, and a gapping portion.

Clause 17: The brassiere of clause 14, wherein: the prosthetic breast form comprises a compressible material such that a volume of the prosthetic breast form can be reduced while being inserted into the pocket.

Clause 18: The brassiere of clause 14, wherein: the prosthetic breast form comprises a percent mass of the total prosthetic brassier and said percent mass is 50%-70%.

Clause 19: The brassiere of clause 14, wherein: the band lacks a fastening mechanism.

Clause 20: The brassiere of clause 14, wherein: the front panel, the first shoulder strap, the second shoulder strap, and the band comprise a textile material; and the textile material is the same for each the front panel, the first shoulder strap, the second shoulder strap, and the band.

Clause 21: The brassiere of clause 14, wherein: the front panel further comprises a lace panel.

Clause 22: The brassiere of claim 21, wherein: the front panel is coupled to the inner layer on a side distal from the user's body.

Clause 23: The brassiere of claim 14, wherein: the front panel lacks a vertical bifurcating mechanism.

Clause 24: The brassiere of clause 14, wherein: the front panel further comprises a neckline with a shape chosen from a group consisting of square, V-neck, scoop, or sweetheart.

Clause 25: The brassiere of clause 14, wherein: the prosthetic breast form comprises a flat bottom portion.

What is claimed is:

1. A brassiere comprising:
a front panel;
a band;
a first shoulder strap;
a second shoulder strap; and
a prosthetic breast form;
wherein:
the front panel further comprises an outer layer and an inner layer joined along a first portion of a perimeter edge;
the outer layer and inner layer are permanently joined along the first portion of the perimeter edge and define a pocket configured to receive the prosthetic breast form;
the outer layer and inner layer are selectively fastenable along a second portion of the perimeter edge to define an access slot to the pocket;
wherein:
the band is configured to be non-removably joined to the front panel under each of a user's armpits and wrap around the user's back; and
each of the first and second shoulder straps are joined to both the front panel and band and configured to extend over each of the user's shoulders.

2. The brassiere of claim 1, wherein:
the prosthetic breast form comprises a first cup, a second cup, and a gapping portion.

3. The brassiere of claim 1, wherein:
the prosthetic breast form comprises a percent mass of the total brassiere and said percent mass is 50%-70%.

4. The brassiere of claim 1, wherein:
the band lacks a fastening mechanism.

5. The brassiere of claim 1, wherein:
the front panel lacks a vertical bifurcating mechanism.

6. The brassiere of claim 1, wherein:
a length of the access slot is between 4.0 inches and 7.0 inches.

7. The brassiere of claim 1, wherein:
the front panel, the first shoulder strap, the second shoulder strap, and the band comprise a textile material; and
the textile material is the same for each the front panel, the first shoulder strap, the second shoulder strap, and the band.

8. The brassiere of claim 1, wherein:
the prosthetic breast form can be inserted into the pocket while the brassiere is being worn by the user.

9. The brassiere of claim 1, wherein:
the prosthetic breast form comprises a compressible material such that a volume of the prosthetic breast form can be reduced while being inserted into the pocket.

10. The brassiere of claim 1, wherein:
the front panel further comprises a lace panel.

11. The brassiere of claim 10, wherein:
the front panel is coupled to the inner layer on a side distal from the user's body.

12. A brassiere comprising:
a front panel;
a band;
a first shoulder strap;
a second shoulder strap; and
a prosthetic breast form;
wherein:
the band is non-removably joined to the front panel under each of a user's armpits and wraps around the users body such that the front panel is adjacent to the user's chest;
the front panel further comprises an outer layer and an inner layer joined along a first portion of a perimeter edge;
the outer layer and inner layer are permanently joined along the first portion of the perimeter edge and define a pocket configured to receive the prosthetic breast form;
the outer layer and inner layer are not joined along a second portion of the perimeter edge to define an access slot to the pocket; and
wherein the prosthetic breast form can be inserted into the pocket through the access slot while the user is wearing the brassiere.

13. The brassiere of claim 12, wherein:
the prosthetic breast form comprises a single cup.

14. The brassiere of claim 12, wherein:
the prosthetic breast form comprises a first cup, a second cup, and a gapping portion.

15. The brassiere of claim 12, wherein:
the prosthetic breast form comprises a compressible material such that a volume of the prosthetic breast form can be reduced while being inserted into the pocket.

16. The brassiere of claim 12, wherein:
the prosthetic breast form comprises a percent mass of the total prosthetic brassier and said percent mass is 50%-70%.

17. The brassiere of claim 12, wherein:
the band lacks a fastening mechanism.

18. The brassiere of claim 12, wherein:
the front panel, the first shoulder strap, the second shoulder strap, and the band comprise a textile material; and
the textile material is the same for each the front panel, the first shoulder strap, the second shoulder strap, and the band.

19. The brassiere of claim 12, wherein:
the front panel further comprises a lace panel.

20. The brassiere of claim 19, wherein:
the front panel is coupled to the inner layer on a side distal from the user's body.
